(19) United States
(12) Patent Application Publication
(10) Pub. No.: US 2008/0058554 A1
(43) Pub. Date: Mar. 6, 2008

(54) Propyl Bromide Compositions

(75) Inventors: Keith G. Anderson, Baton Rouge, LA (US); James E. Torres, Clinton, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/846,420

(22) Filed: Aug. 28, 2007

Related U.S. Application Data
(60) Provisional application No. 60/824,015, filed on Aug. 30, 2006.
(60) Provisional application No. 60/953,013, filed on Jul. 31, 2007.

(51) Int. Cl. C07C 49/00

(52) U.S. Cl. 568/414

(57) ABSTRACT

This invention provides wet and dry n-propyl bromide compositions comprising minimal amounts of acetone, bromoacetone, 1-propanol, and isopropyl bromide. These n-propyl bromide compositions are formed by free-radical catalyzed hydrobromination of propylene with hydrogen bromide.

### REFERENCE TO RELATED APPLICATIONS

This application claims the benefit and priority of U.S. Provisional Application No. 60/824,015, filed Aug. 30, 2006, and U.S. Provisional Application No. 60/953,013, filed Jul. 31, 2007, the disclosures of which are incorporated herein by reference.

### TECHNICAL FIELD

This invention relates to novel crude product and dry n-propyl bromide compositions.

### BACKGROUND

n-Propyl bromide (also referred to as 1-bromopropane or propyl bromide) is a commercially useful product, in particular as a degreasing agent, and in cleaning solutions for electrical circuit board production. In such applications, the presence of certain impurities in the n-propyl bromide is undesirable. Thus, it would be advantageous to produce n-propyl bromide having fewer impurities initially, so that if further purification is desired, the amount of work to purify the n-propyl bromide to the desired level is thereby minimized.

Methods for producing n-propyl bromide are known. One such general method involves hydrobromination of propylene using free-radical catalysis. Commonly-owned copending applications (U.S. Patent Application No. 60/673,391, filed Apr. 18, 2005, and its corresponding PCT application, Application No. PCT/US2006/13778, filed Apr. 13, 2006, claiming priority thereof, U.S. Patent Application No. 60/791,850, filed Apr. 13, 2006, and its corresponding PCT application, Application No. PCT/US2007/66409, filed Apr. 11, 2007, claiming priority thereof; and U.S. Patent Application No. 60/803,833, filed Jun. 2, 2006, and its corresponding PCT application, Application No. PCT/US2007/70168, filed Jun. 1, 2007, claiming priority thereof) describe preferred hydrobromination reactions of this type. For example, the process of Application No. 60/791,850 as applied to n-propyl bromide production comprises continuously feeding propylene, gaseous hydrogen bromide, and a molecular oxygen-containing gas into a liquid phase reaction medium comprised of aliphatic bromide (preferably n-propyl bromide, corresponding to the product being produced) to cause anti-Markovnikov addition of HBr to propylene, the feeds being proportioned and maintained to provide a molar excess of hydrogen bromide relative to propylene in the range of about 1 to about 5 percent, and a molar ratio of molecular oxygen to propylene of less than 0.005, and preferably in the range of about 0.00005:1 to about 0.001:1. The principal product is n-propyl bromide with small amounts of isopropyl bromide also being co-formed.

Studies in our laboratories have shown that formation of trace amounts of bromine and peroxy radicals are formed during the hydrobromination reaction. This in turn leads to the formation of small amounts of various other by-products such as 1,2-dibromopropane, acetone, bromoacetone, propionaldehyde, 1,3-dibromoacetone, 1-propanol, and 2-propanol. The formation of acetone and bromoacetone, typically at levels of about 200 ppm, is especially undesirable as bromoacetone, even at such low concentrations, is a powerful lachrymator.

Conventional distillation procedures can be used to purify propyl bromide products formed by the hydrobromination of propylene. However, such distillations typically require expensive distillation facilities operated under close control of conditions if highly pure n-propyl bromide is to be recovered.

### SUMMARY OF THE INVENTION

Pursuant to this invention, n-propyl bromide compositions with very few impurities can be obtained from n-propyl bromide formed by free-radical catalyzed hydrobromination of propene. One advantage of the production of mixtures having fewer impurities is that less waste is produced, minimizing the amount of material for disposal.

One embodiment of this invention is a reaction-derived n-propyl bromide composition which is a crude product. This composition comprises

i) about 90 GC area % or more of n-propyl bromide,

ii) about 50 ppm or less of acetone,

iii) about 20 ppm or less of bromoacetone,

iv) less than about 20 ppm of 1-propanol, and

v) about 100,000 ppm or less of isopropyl bromide.

### The reaction-derived crude product n-propyl bromide composition can comprise

i) about 90 GC area % or more of n-propyl bromide,

ii) about 1 ppm to about 50 ppm of acetone,

iii) about 0.10 ppm to about 20 ppm of bromoacetone,

iv) about 1 ppm to less than about 20 ppm of 1-propanol, and

v) about 1 ppm to about 100,000 ppm of isopropyl bromide.

The crude product is formed by free-radical catalyzed hydrobromination of propylene with hydrogen bromide to form a crude product. Such crude product n-propyl bromide compositions are referred to herein, including the claims, as reaction-derived n-propyl bromide compositions. These compositions are also commonly referred to as the crude reaction product.

Another embodiment of this invention is a reaction-derived n-propyl bromide composition which is a dry organic phase. This composition comprises

i) about 90 GC area % or more of n-propyl bromide,

ii) about 50 ppm or less of acetone,

iii) about 20 ppm or less of bromoacetone,

iv) about 50 ppm or less of 1-propanol, and

v) about 100,000 ppm or less of isopropyl bromide.

### The reaction-derived dry organic phase n-propyl bromide composition can comprise

i) about 90 GC area % or more of n-propyl bromide,

ii) about 1 ppm to about 50 ppm of acetone,

iii) about 0.10 ppm to about 20 ppm of bromoacetone,

iv) about 1 ppm to about 50 ppm of 1-propanol, and

v) about 1 ppm to about 100,000 ppm of isopropyl bromide.

The dry organic phase is formed by free-radical catalyzed hydrobromination of propylene with hydrogen bromide to form a crude product, which crude product is subjected to water washing, formation therefrom of an aqueous phase and an organic phase comprising propyl bromide, physical separation of the aqueous phase from the organic phase, and drying of said organic phase. Such dried n-propyl bromide compositions are referred to herein, including the claims, as dry reaction-derived n-propyl bromide compositions. These compositions are also commonly referred to as dry crude product.

Still another embodiment of this invention is an n-propyl bromide composition. This composition comprises

i) about 99.90 GC area % or more of n-propyl bromide,

ii) about 20 ppm or less of acetone,

iii) less than about 10 ppm of bromoacetone,

iv) less than about 20 ppm of 1-propanol, and

v) about 1000 ppm or less of isopropyl bromide.

### The n-propyl bromide composition can comprise

i) about 99.90 GC area % or more of n-propyl bromide,

ii) about 1 ppm to about 20 ppm of acetone,

iii) about 0.10 ppm to less than about 10 ppm of bromoacetone,

iv) about 1 ppm to less than about 20 ppm of 1-propanol, and

v) about 1 ppm to about 1000 ppm of isopropyl bromide.

### The n-propyl bromide composition is formed by free-radical catalyzed hydrobromination of propylene with hydrogen bromide.

These and other embodiments and features of this invention will be still further apparent from the ensuing description and appended claims.

### FURTHER DETAILED DESCRIPTION

As used herein, including the claims, and unless expressly indicated otherwise, the term “crude propyl bromide” refers to n-propyl bromide in admixture with co-products and/or impurities resulting from preparation by free-radical catalyzed hydrobromination of propylene with hydrogen bromide. The term “crude propyl bromide” does not denote, imply, or suggest that the crude propyl bromide must be purified before use.

The term “reaction-derived” means that the composition of the product is reaction determined and not the result of use of downstream purification techniques, such as recrystallization or chromatography, or like procedures that can affect the chemical composition of the product. In other words, the products of such high purity are directly produced in the synthesis process apart from use of subsequent purification procedures (other than simple washing steps) as applied to the recovered or isolated products.

The abbreviation ppm means parts per million based on GC area percent, unless specifically stated otherwise herein.

It has been discovered that use of feeds of molecular oxygen in amounts that are very low enable the hydrobromination of propylene to proceed efficiently and with minimization or suppression of undesirable by-product formation, thereby enabling recovery of highly pure n-propyl bromide. Such a process is described in more detail in U.S. Patent Application No. 60/791,850, which is hereby incorporated by reference. For a preferred separation and purification process, see U.S. Patent Application No. 60/803,833, which is hereby incorporated by reference.

The crude product n-propyl bromide composition is directly formed in the hydrobromination reactor; no purification steps have been performed on the crude product. Although unpurified, the reaction-derived crude product n-propyl bromide composition comprises mainly n-propyl bromide together with organic coproducts and/or impurities. Generally, the only substances present in the crude product n-propyl bromide composition are substances that are formed during the formation of the crude product n-propyl bromide composition.

Even though the crude product has not been purified, this reaction-derived n-propyl bromide composition is relatively pure. The crude product n-propyl bromide composition comprises i) about 90 GC area % or more of n-propyl bromide, ii) about 25 ppm or less of acetone, iii) about 20 ppm or less of bromoacetone, iv) less than about 20 ppm of 1-propanol, and v) about 100,000 ppm or less of isopropyl bromide. The crude product n-propyl bromide composition can comprise i) about 90 GC area % or more of n-propyl bromide, ii) about 1 ppm to about 50 ppm of acetone, iii) about 0.10 ppm to about 20 ppm of bromoacetone, iv) about 1 ppm to less than about 20 ppm of 1-propanol, and v) about 1 ppm to about 100,000 ppm of isopropyl bromide.

In preferred embodiments, the crude product n-propyl bromide composition comprises about 95 GC area % or more of n-propyl bromide. The amount of acetone is preferably about 30 ppm or less, more preferably about 25 ppm or less, still more preferably about 20 ppm or less. In especially preferred crude product compositions, the amount of acetone is about 15 ppm or less, more preferably about 10 ppm or less. Crude products with amounts of acetone as low as about 5 ppm have been obtained. Bromoacetone is preferably about 10 ppm or less, and more preferably about 5 ppm or less. There is preferably less than about 10 ppm 1-propanol, and more preferably less than about 5 ppm 1-propanol in the crude product n-propyl bromide composition. Crude products with no detectable 1-propanol have been obtained. It is preferred that the crude product has about 50,000 ppm isopropyl bromide or less, and more preferably about 25,000 ppm isopropyl bromide or less. Amounts of isopropyl bromide of about 16,500 ppm or less have been obtained in the crude product n-propyl bromide compositions of this invention.

In particularly preferred embodiments, there is about 95 GC area % or more of n-propyl bromide, and about 30 ppm acetone or less, more preferably about 25 ppm acetone or less, in the crude product n-propyl bromide composition. Still more preferred are crude product compositions having about 95 GC area % or more of n-propyl bromide, and about 20 ppm acetone or less, more preferably about 15 ppm acetone or less. In other particularly preferred embodiments, the crude product n-propyl bromide composition has about 95 GC area % or more of n-propyl bromide, and about 10 ppm or less of bromoacetone. An especially preferred embodiment is a crude product n-propyl bromide composition in which there is about 97 GC area % or more of n-propyl bromide, about 30 ppm or less of acetone, and about 5 ppm or less of bromoacetone. Still more preferably, less than about 10 ppm of 1-propanol and/or about 25,000 ppm or less of isopropyl bromide are present in these particularly preferred embodiments.

The dry product n-propyl bromide composition is formed directly from the crude product by subjecting the crude product from the hydrobromination to water washing and drying. Once the crude product has been water-washed, the water-washed mixture is allowed to form an (upper) aqueous phase and a (lower) organic phase. The aqueous phase and the organic phase are physically separated from each other. The separated organic phase is the a wet reaction-derived n-propyl bromide phase. There is no distillation or other purification step, such as column chromatography, that is performed on the crude product or the washed organic phase to form the wet n-propyl bromide phase. This composition phase is wet because it usually retains some of the water (˜500 ppm) from the water wash. Generally, the only substances present in the dry product n-propyl bromide composition are substances that are formed during the formation of the dry product n-propyl bromide composition and water.

Drying of the wet n-propyl bromide phase to directly form the dry reaction-derived n-propyl bromide composition can be accomplished by heating the wet n-propyl bromide phase to one or more temperatures in the range of about 67° C. to about 72° C., at which temperature(s) the water will boil away from the n-propyl bromide. When (most of) the water has been removed, the dry reaction-derived n-propyl bromide composition is obtained. In other words, as used herein, there is no other purification step, such as column chromatography, that is performed on the wet n-propyl bromide phase to form the dry reaction-derived n-propyl bromide composition. This composition is often contains some water, usually about 150 ppm or less, and amounts of water as low as about 1 ppm have been achieved. The dry reaction-derived n-propyl bromide composition comprises mainly n-propyl bromide together with organic coproducts and/or impurities.

Staged stripping is one drying method that can form the dry reaction-derived n-propyl bromide composition of this invention. When the drying is accomplished by stripping, the water comes out in the distillate because it is stripped by the organic vapor in the stripper column. HBr and acetone are more volatile than the water; thus, the levels of water, acetone, and HBr are also reduced when the water is removed by stripping.

Even though the only treatment of the wet reaction-derived n-propyl bromide composition has been drying to form a dry reaction-derived n-propyl bromide composition, this dry reaction-derived n-propyl bromide composition is also quite pure, and thus does not need to be purified before use. The dry reaction-derived n-propyl bromide composition comprises i) about 90 GC area % or more of n-propyl bromide, ii) about 25 ppm or less of acetone, iii) about 20 ppm or less of bromoacetone, iv) about 50 ppm or less of 1-propanol, and v) about 100,000 ppm or less of isopropyl bromide. The dry reaction-derived n-propyl bromide composition can comprise i) about 90 GC area % or more of n-propyl bromide, ii) about 1 ppm to about 50 ppm of acetone, iii) about 0.10 ppm to about 20 ppm of bromoacetone, iv) about 1 ppm to about 50 ppm of 1-propanol, and v) about 1 ppm to about 100,000 ppm of isopropyl bromide. Amounts of water as low as about 5 ppm have been obtained in the dry reaction-derived n-propyl bromide compositions of this invention.

In preferred embodiments, dry reaction-derived n-propyl bromide compositions comprise about 95 GC area % or more of n-propyl bromide. The amount of acetone is preferably about 30 ppm or less, and more preferably is about 20 ppm or less. The amount of acetone is preferably about 30 ppm or less, more preferably about 25 ppm or less, still more preferably about 20 ppm or less. In especially preferred dry reaction-derived n-propyl bromide compositions, the amount of acetone is about 15 ppm or less, more preferably about 10 ppm or less. Amounts of acetone as low as about 3 ppm have been obtained. Bromoacetone is preferably about 10 ppm or less, and more preferably about 5 ppm or less. There is preferably about 25 ppm or less of 1-propanol, and more preferably about 15 ppm or less of 1-propanol in the dry reaction-derived n-propyl bromide composition. 1-Propanol amounts of less than about 10 ppm have been obtained. It is preferred that the dry reaction-derived n-propyl bromide composition has about 50,000 ppm or less of isopropyl bromide, and more preferably about 25,000 ppm or less of isopropyl bromide.

In particularly preferred embodiments, there is about 95 GC area % or more of n-propyl bromide, and about 30 ppm or less, more preferably about 25 ppm acetone or less, in the dry reaction-derived n-propyl bromide composition. Still more preferred are crude product compositions having about 95 GC area % or more of n-propyl bromide, and about 20 ppm acetone or less, more preferably about 15 ppm acetone or less. In other particularly preferred embodiments, the dry reaction-derived n-propyl bromide composition has about 95 GC area % or more of n-propyl bromide, and less than about 10 ppm of bromoacetone. An especially preferred embodiment is a dry reaction-derived n-propyl bromide composition in which there is about 97 GC area % or more of n-propyl bromide, about 30 ppm or less of acetone, and about 10 ppm or less of bromoacetone. Still more preferably, about 25 ppm or less of 1-propanol and/or about 25,000 ppm or less of isopropyl bromide are present in these particularly preferred embodiments.

Product purification can be carried out using distillation procedures which are not of this invention. For example, separation of isopropyl bromide and other impurities from n-propyl bromide can be achieved by further distillation(s).

After such further purification, n-propyl bromide purities of 99.90% are achievable. Thus, this invention also provides n-propyl bromide compositions having about 99.90 GC area % or more of n-propyl bromide, about 20 ppm or less of acetone, less than about 10 ppm of bromoacetone, less than about 20 ppm of 1-propanol, and about 1000 ppm or less of isopropyl bromide. The n-propyl bromide compositions can comprise about 99.90 GC area % or more of n-propyl bromide, about 1 ppm to about 20 ppm of acetone, about 0.10 ppm to less than about 10 ppm of bromoacetone, about 1 ppm to less than about 20 ppm of 1-propanol, and about 1 ppm to about 1000 ppm of isopropyl bromide. Generally, the only substances present in the n-propyl bromide composition that has been further purified are substances that are formed during the formation of the n-propyl bromide, and water.

Preferred n-propyl bromide compositions have about 99.92 GC area % or more of n-propyl bromide. Compositions having about 99.96 GC area % or more of n-propyl bromide have been obtained. The amount of acetone is preferably about 15 ppm or less, and more preferably is about 10 ppm or less. Amounts of acetone as low as 1 ppm have been obtained. Bromoacetone is preferably less than about 5 ppm, and more preferably less than about 3 ppm of the n-propyl bromide composition. N-propyl bromide compositions with no detectable bromoacetone have been obtained. There is preferably less than about 10 ppm of 1-propanol, and more preferably less than about 5 ppm of 1-propanol in the n-propyl bromide composition. N-propyl bromide compositions with no detectable 1-propanol have been obtained. It is preferred that the n-propyl bromide composition has about 800 ppm or less of isopropyl bromide, and more preferably about 400 ppm or less of isopropyl bromide.

In particularly preferred embodiments, there is about 99.92 GC area % or more of n-propyl bromide, and about 15 ppm or less of acetone in the n-propyl bromide composition. In other particularly preferred embodiments, the n-propyl bromide composition has about 99.92 GC area % or more of n-propyl bromide, less than about 5 ppm bromoacetone, and about 15 ppm or less of acetone. Still more preferably, less than about 10 ppm of 1-propanol and/or about 800 ppm or less of isopropyl bromide are present in these particularly preferred embodiments.

The crude product n-propyl bromide compositions of this invention and dry reaction-derived n-propyl bromide compositions of this invention are useful without further purification, e.g., in metal cleaning, where very pure solvents are not usually required. All of the compositions of this invention can be subjected to further purification, if desired.

If desired, stabilizers and/or other additives may be added to the n-propyl bromide compositions of this invention. However, it is preferred, especially for the crude product n-propyl bromide composition and the dry reaction-derived n-propyl bromide composition, that no stabilizer is present in the composition.

### Analytical Procedures

### Sample Preparation

The sample preparation procedure depended on the source of the sample. From the reaction-derived crude product n-propyl bromide composition, the initial solution had two phases; the bottom phase was dried with calcium chloride prior to analysis. The dry product n-propyl bromide composition was used as a control (i.e., it was injected into GC1 A-side, see below). Dry product n-propyl bromide composition samples and samples of further purified n-propyl bromide compositions were treated with calcium chloride pellets and filtered through a cotton-packed glass pipette prior to gas chromatography analysis.

When it was suspected that a sample might contain HBr, calcium chloride pellets (2 grams) were placed in a scintillation vial, sample (10 mL) was added to the vial, the vial was capped and then shaken until sample was clear in color. The sample for analysis was then taken from the vial.

When a sample had a relatively high amount of HBr present (indicated by an orange or reddish color), 5 mL of the sample were placed in a scintillation vial. Deionized water (5 mL) was then added to the vial, the vial was capped and then shaken until the orange or reddish color disappeared. The vial was allowed to sit. Two layers formed; the top layer was discarded. To another scintillation vial, calcium chloride pellets (2 grams) were added, and then ˜4 mL of the bottom layer from the first vial were added to the vial containing the calcium chloride, the vial was capped and then shaken for a few moments. A cotton-packed glass pipette was placed in a third scintillation vial. The sample from the second scintillation vial was transferred into the third scintillation vial through the cotton-plugged pipette. The sample for analysis was then taken from the third scintillation vial.

### Sample Analysis

Samples were analyzed by gas chromatography (GC) on an Agilent 6890N Network GC System gas chromatograph with a 30 M×0.32 mm×1.8 um column (J & W Scientific). The GC procedure utilized a two-staged, ramped temperature program for the GC column. The sample was injected into the column at an initial column temperature of 40° C. and held at this temperature for 8 minutes. The column temperature was then increased at a rate of 5° C. per minute until the temperature reached 70° C. The rate of temperature increase was then changed to 15° C. per minute until a final column temperature of 250° C. was reached. This final column temperature was held for 5 minutes. Some of the operating parameters are listed in Table 1.

TABLE 1

| Parameter | Value |
|---|---|
| Injection type | automatic |
| Injection volume | 0.5 μL |
| Injector temperature | 150° C. |
| Flow rate | 2.0 mL per minute |
| Velocity | 33 mL per minute |
| Injector split ratio | 11:2 |
| Split flow | 11 mL per minute |
| Total flow | 15.6 mL per minute |
| Detector type | flame ionization detector |
| | (FID) |
| Detector temperature | 250° C. |
| Detector Hydrogen Flow | 40 mL per minute |
| Detector air flow rate | 450 mL per minute |
| Make-up gas (helium) flow | 50 mL per minute |
| rate | |
| Mode | constant make-up |

The samples were transferred into GC autosampler vials, where the GC syringe filled and discarded three portions of each sample before injecting a sample for analysis into the injection port while simultaneously initiating the two-stage GC column temperature program and the data acquisition computer program, which was set to report data as GC area percent of sample components. The GC autosampler clean-up routine consisted of four sets of filling the syringe with acetone and discarding the syringe contents, followed by four sets of filling the syringe with pentane and discarding the syringe contents.

It is to be understood that chemicals referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

Except as may be expressly otherwise indicated, the article “a” or “an” if and as used herein is not intended to limit, and should not be construed as limiting, a claim to a single element to which the article refers. Rather, the article “a” or “an” if and as used herein is intended to cover one or more such elements, unless the text expressly indicates otherwise.

Each and every patent, publication, or commonly-owned patent application referred to in any portion of this specification is incorporated in toto into this disclosure by reference, as if fully set forth herein.

This invention is susceptible to considerable variation in its practice. Therefore the foregoing description is not intended to limit, and should not be construed as limiting, the invention to the particular exemplifications presented hereinabove.

That which is claimed is:

1. A reaction-derived n-propyl bromide composition which is a crude product comprising
i) about 90 GC area % or more of n-propyl bromide,
ii) about 25 ppm or less of acetone,
iii) about 20 ppm or less of bromoacetone,
iv) less than about 20 ppm of 1-propanol, and
v) about 100,000 ppm or less of isopropyl bromide,
wherein said crude product is formed by free-radical catalyzed hydrobromination of propylene with hydrogen bromide.

2. A composition as in claim 1 which comprises
i) about 90 GC area % or more of n-propyl bromide,
ii) about 1 ppm to about 50 ppm of acetone,
iii) about 0.10 ppm to about 20 ppm of bromoacetone,
iv) about 1 ppm to less than about 20 ppm of 1-propanol, and
v) about 1 ppm to about 100,000 ppm of isopropyl bromide.

3. A composition as in claim 1 which has about 95 GC area % or more of n-propyl bromide.

4. A composition as in claim 1 which has at least one of the following features:
about 30 ppm or less of acetone;
about 10 ppm or less of bromoacetone;
about 10 ppm or less of 1-propanol;
about 50,000 ppm or less of isopropyl bromide.

5. A composition as in claim 1 which has about 95 GC area % of n-propyl bromide, and which has about 30 ppm or less of acetone.

6. A composition as in claim 1 which has about 95 GC area % of n-propyl bromide, and which has about 10 ppm or less of bromoacetone.

7. A composition as in claim 1 which has about 97 GC area % of n-propyl bromide, which has about 30 ppm or less of acetone, and which has about 10 ppm or less of bromoacetone.

8. A composition as in claim 1 wherein the only substances present in said crude product are substances that are formed during the formation of said composition.

9. A reaction-derived n-propyl bromide composition which is a dry organic phase comprising
i) about 90 GC area % or more of n-propyl bromide,
ii) about 25 ppm or less of acetone,
iii) about 20 ppm or less of bromoacetone,
iv) about 50 ppm or less of 1-propanol, and
v) about 100,000 ppm or less of isopropyl bromide,
wherein said dry organic phase is formed by free-radical catalyzed hydrobromination of propylene with hydrogen bromide to form a crude product, which crude product is subjected to water washing, formation therefrom of an aqueous phase and an organic phase comprising propyl bromide, physical separation of the aqueous phase from the organic phase, and drying of said organic phase.

10. A composition as in claim 9 which comprises
i) about 90 GC area % or more of n-propyl bromide,
ii) about 1 ppm to about 50 ppm of acetone,
iii) about 0.10 ppm to about 20 ppm of bromoacetone,
iv) about 1 ppm to about 50 ppm of 1-propanol, and
v) about 1 ppm to about 100,000 ppm of isopropyl bromide.

11. A composition as in claim 9 which has about 95 GC area % or more of n-propyl bromide.

12. A composition as in claim 9 which has at least one of the following features:
about 30 ppm or less of acetone;
about 10 ppm or less of bromoacetone;
about 25 ppm or less of 1-propanol;
about 90,000 ppm or less of isopropyl bromide.

13. A composition as in claim 9 which has about 95 GC area % or more of n-propyl bromide, and which has about 30 ppm or less of acetone.

14. A composition as in claim 9 which has about 95 GC area % or more of n-propyl bromide, and which has about 10 ppm or less of bromoacetone.

15. A composition as in claim 9 which has about 97 GC area % or more of n-propyl bromide, which has about 30 ppm or less of acetone, and which has about 10 ppm or less of bromoacetone.

16. A composition as in claim 9 wherein the only substances present in said dry product n-propyl bromide composition are substances that are formed during formation of said composition and water.

17. An n-propyl bromide composition which comprises
i) about 99.90 GC area % or more of n-propyl bromide,
ii) about 20 ppm or less of acetone,
iii) less than about 10 ppm of bromoacetone,
iv) less than about 20 ppm of 1-propanol, and
v) about 1000 ppm or less of isopropyl bromide,
wherein said composition is formed by free-radical catalyzed hydrobromination of propylene with hydrogen bromide.

18. A composition as in claim 17 which comprises
i) about 99.90 GC area % or more of n-propyl bromide,
ii) about 1 ppm to about 20 ppm of acetone,
iii) about 0.10 ppm to less than about 10 ppm of bromoacetone,
iv) about 1 ppm to less than about 20 ppm of 1-propanol, and
v) about 1 ppm to about 1000 ppm of isopropyl bromide.

19. A composition as in claim 17 which has about 99.92 GC area % or more of n-propyl bromide.

20. A composition as in claim 17 which has at least one of the following features:
about 15 ppm or less of acetone;
less than about 5 ppm of bromoacetone;
less than about 10 ppm of 1-propanol;
about 800 ppm or less of isopropyl bromide.

21. A composition as in claim 17 which has about 99.92 GC area % or more of n-propyl bromide, and which has about 15 ppm or less of acetone.

22. A composition as in claim 17 which has about 99.92 GC area % or more of n-propyl bromide, and which has less than about 5 ppm of bromoacetone.

23. A composition as in claim 17 which has about 99.92 GC area % or more of n-propyl bromide, which has about 15 ppm or less of acetone, and which has less than about 5 ppm of bromoacetone.

24. A composition as in claim 23 which has about 800 ppm or less of isopropyl bromide.

25. A composition as in claim 17 wherein the only substances present in said n-propyl bromide composition are substances that are formed during the formation of the n-propyl bromide, and water.
